(19) United States
(12) Patent Application Publication
(10) Pub. No.: US 2002/0188253 A1
(43) Pub. Date: Dec. 12, 2002

(54) Method and apparatus for drug delivery in veins

(75) Inventors: Lucas S. Gordon, Sammamish, WA (US); Mary Jo Gordon, Carnation, WA (US); Robert C. Lichty II, Santa Rosa, CA (US); John J. Thomas, Bothell, WA (US)

Correspondence Address: BOARD OF DIRECTORS, PHARMASPEC CORPORATION, 7901 168TH AVENUE, SUITE 102, REDMOND, WA, 98052, US

(73) Assignee: Pharmaspec Corporation

(21) Appl. No.: 09/877,396

(22) Filed: Jun. 7, 2001

(51) Int. Cl. A61M 29/00; A61M 31/00

(52) U.S. Cl. 604/101.03; 604/509; 600/470

(57) ABSTRACT

Methods and apparatus for creating and delivering a medical agent into an isolated vein segment, the isolated vein segment not having any communicating vein branches, the apparatus comprising a catheter having at least expandable vein occlusion devices to engage and expand the inside wall of a vein, thereby blocking the interior of the vein and preventing fluid located in the isolated segment from flowing between the occlusion devices and the wall of the vein at a pressure within the isolated vein segment of at least 100, and preferably 200, millimeters of mercury, the catheter including one or more lumens for directing a fluid into the isolated vein segment at a pressure of at least 100 millimeters of mercury.

### FIELD OF THE INVENTION

The present invention pertains to methods and apparatus for delivering medical agents in veins, and more particularly to the delivery of medical agents in isolated vein segments.

### BACKGROUND OF THE INVENTION

In most areas of the human body, including the limbs, veins may be classified by function as either “returning” veins or “collecting” veins. Returning veins are typically 5 to 25 millimeters in diameter, while collecting veins are only ½ to 5 millimeters in diameter. The collecting veins serve to “collect” the smaller veins and venules that lead from the capillaries. Returning veins provide a conduit for flow of blood back to the heart. In most areas of the body, both returning and collecting veins are interconnected to form a grid of parallel paths flowing to the heart.

Veins that provide parallel flow paths to the heart branch from both returning veins and collecting veins, and are called “communicating veins.” The collecting veins also have branching veins that do not communicate with the main venous grid, but instead branch out, (e.g., as tree limbs), dividing into smaller vessels that branch into venules, with the venules branching into capillaries. Consequently, retrograde perfusion is ineffective for delivering medical agents to the venules and capillaries in areas of the body where there are multiple communicating veins providing redundant flow paths for returning blood. For example, delivering a fluid retrograde into a vein containing communicating veins merely results in the fluid flowing retrograde to the first communicating vein and then flowing antegrade back toward the heart. No fluid will flow retrograde to the desired treatment area.

U.S. Pat. Nos. 4,689,041 and 5,033,998 issued to Corday et al. describe a method using a catheter carrying an isolating expandable balloon on a distal end for retrograde venous injection of various fluids into a blockaded region of the heart made inaccessible by an occluded artery. The described balloon isolation method involves placing the balloon into the coronary sinus and directing fluid beyond the balloon retrograde into all veins of the heart. In this instance, standard retrograde perfusion works well, because the goal is to deliver cardioplegic solution to the entire heart tissue. While aiding in the retrograde delivery of fluids into the veins of the heart, the balloon isolation method does not provide an effective method or device for delivering medical agents within veins of the body containing communicating veins, whether returning or collecting, since the delivered fluid will flow only to the first communicating vein and then flow back toward the heart—i.e., no fluid will flow retrograde to the venules and capillaries. In these veins there is no defined retrograde or antegrade direction since blood flows in either direction, depending on the pressure gradient at each end of the vessel.

The presence of communicating veins has also complicated the localized delivery of medications to treat diseases of peripheral limbs, e.g., injected medication for diabetic foot ulcers may easily escape the treatment area through communicating veins. Known techniques to overcome this problem include applying a tourniquet around a limb to inflict a pressure above arterial blood pressure, resulting in complete stasis of the circulatory system in the limb. Another known technique is to combine venous injection with circulatory arrest (tourniquet) for injection of a medication mixed with a large volume of liquids into a vein on the dorsum of the foot. The voluminous injection results in expansion and flooding of the entire venous system within the foot and lower leg. Although this technique is effective in healing diabetic foot ulcers, many patients report at least moderate pain during the procedure. Nor is there any known literature describing how this technique could be used for localized drug delivery into the veins draining the ulcerated tissue.

### SUMMARY OF THE INVENTION

In accordance with a general aspect of the invention, methods and apparatus are provided for localized delivery of medical agents in an isolated vein location that does not contain any communicating veins. Towards this end, an invasive device, such as a suitably designed catheter, is inserted and advanced through the venous system into a desired collecting vein segment. A variety of insertion locations are suitable and the catheter device may be advanced in a retrograde or antegrade direction to the vein segment location. A verification procedure is then performed to ensure communicating veins are not present in the selected vein segment.

In one embodiment, the verification process is accomplished by isolating the vein segment using a pair of spaced apart expandable occlusion devices carried on the catheter device. An imaging fluid is then injected through a lumen in the catheter device into the isolated segment, while viewing the isolated segment with a fluoroscope, magnetic resonance imaging system or other suitable imaging modality. The expandable occlusion devices preferably form seals with the vein segment wall that are able to withstand at least a pressure of at least 100, and preferably 200, millimeters of mercury in order to obtain imaging fluid flow through very small communicating veins.

In another embodiment, the verification process is accomplished is accomplished by injecting fluid into the isolated vein segment while measuring a differential pressure within the isolated vein segment. For example, a pressure sensor carried on the catheter device positioned within an isolated vein segment may be used. Alternately, a pressure sensor may be carried on a distal end of the catheter device in fluid communication with a fluid injection lumen. At slow fluid injection rates, the dynamic pressure drop through the catheter is negligible. Thus, as fluid is injected into the isolated vein segment, a differential pressure is measured. By measuring a rise in pressure within the isolated vein segment as fluid is injected, communicating veins may be detected. If larger communicating veins are present, there will be a minimal differential pressure beyond the normal venous pressure of about 5-10 millimeters of mercury. Small or even microscopic communicating veins may also be present and will manifest by showing pressures of about 10 to about 100 millimeters of mercury. If there are no communicating veins, but only serial veins and venules, then there will be a larger rise in pressure within the isolated vein segment as fluid is injected. Since the injected fluid must overcome both the static arterial back pressure and the dynamic pressure drop of the fluid flow within the venules and serial veins, there will be a pressure differential of at least 100 millimeters of mercury, although a measured infusion pressure of about 200 millimeter of mercury is preferred to insure retrograde flow of the medical agent into the venules and capillaries.

Apparatus embodiments constructed in accordance with the present invention generally comprise a catheter device configured for insertion into a patient's venous system and advancing to a position at a desired vein segment location. For some locations, a slidable guide wire, which can be located within a separate lumen of the catheter device, is helpful in selecting the appropriate vein when advancing the catheter. Usually the vein segment location will have an internal diameter of 4 millimeters or less. The catheter is configured to isolate the vein segment at the desired vein location, and preferably at a location where the vein segment has only serial vein or venule side branches.

In one embodiment, a catheter device incorporates at least two occlusion devices (e.g., expandable balloons) to engage the veins wall of the desired vein segment to thereby block the interior of the vein and prevent fluid from flowing past the occlusion devices at a pressure of at least 100, and preferably at least 200, millimeters of mercury. The catheter device also incorporates a lumen to direct a medical agent into the isolated vein segment and serial vein or venule side branches.

In one embodiment, a catheter device is configured with a pressure measuring for detecting pressure within the isolated vein segment. For example, a pressure sensor may be located between the two expandable vein occlusion devices. Alternately, a pressure measuring device may be located external to the catheter, but in fluid communication with the medical agent infusion lumen. By measuring the pressure within the isolated vein segment, communicating veins within the isolated vein segment may be detected without the use of fluoroscopy. Alternately, by measuring the pressure within the isolated segment, the absence or presence of communicating veins as detected by fluoroscopy may be verified.

In another embodiment, a catheter device carries at least three vein occlusion devices configured to engage the wall of a desired vein location, to thereby isolate at least two separate vein segments. The catheter device also incorporates at least one lumen to direct a medical agent into each of the isolated vein segments. By forming multiple vein segments, this embodiment increases the probability that at least one segment will not contain any communicating veins. Other embodiments may include more than three vein occlusion devices. Factors that may influence the number of vein occlusion devices used in a particular embodiment may include, without limitation, the number of communicating veins in the treatment area, the size of the treatment area, and the desired number of isolated vein segments.

In one embodiment, the occlusive devices each comprise substantially elastic expandable balloons formed of a material with a hardness of between Shore 25D and 55D, and preferably between Shore 35D and 45D. In another embodiment, the substantially elastic expandable balloons comprise a material with a flexural modulus of elasticity of between 500 and 2500 pounds per square inch (psi), and preferably between 1500 and 2000 psi. In still another embodiment, a wall thickness of the substantially elastic expandable balloon is between 0.0005 and 0.0012 inches. In one embodiment, the substantially elastic expandable balloon is attached to, and incorporates a twist along, an axis of the catheter shaft. The twist provides for a tightly wrapped condition of the balloon upon deflation, thus providing a very low profile, allowing the catheter to more easily advance to and withdraw from very small veins. In one embodiment, the substantially elastic expandable balloon is twisted along the axis of the catheter shaft at a twist angle of about 20 degrees.

In certain embodiments, the catheter device may be equipped with a forward looking transducer or imaging device to help navigate though the venous system, whereby detecting valves or other obstructions that are blocking the pathway of the catheter.

In certain embodiments, the catheter device may be equipped with a measuring system that measures the distance the catheter has traveled within the vein. For example, the catheter may have an externally marked graduated scale whereby the distance the catheter has traveled in the vein is measured as the catheter is advanced though the venous system. The measuring system may be used to record the relative positions of valves, communicating veins, desired vein locations, and other locations within the venous system that would be desirable to relocate.

In certain embodiments, the catheter device is equipped with one or more radiopaque markers, visible by x-ray to identify the isolated vein segment and a distal tip of the catheter. Preferably, the radiopaque markers are located within each pair of expandable vein occlusion devices to identify the location of the isolated vein segments. A radiopaque marker on the distal tip of the catheter device will also facilitate navigating through the venous system, including crossing valves and navigating past bifurcations within the venous system. Alternately, ferromagnetic or superparamagnetic markers, visible by magnetic resonance imaging can be used to identify isolated vein segments and/or the distal tip of the catheter.

In certain embodiments, a catheter device incorporates at least two slidebly adjustable occlusion devices (e.g., expandable balloons) in order to facilitate positioning of the occlusion devices at the desired vein segment location.

Other objects and features of the present invention will become apparent hereinafter.

### BRIEF DESCRIPTION OF THE DRAWINGS

The drawings illustrate both the design and utility of preferred embodiments of the present invention, in which similar elements in different embodiments are referred to by the same reference numbers for purposes of ease in illustration of the invention, wherein:

FIG. 1 is a cross-sectional view of a vein having a catheter and introducer sheath inserted therein, wherein two substantially elastic expandable balloons carried on an end of the catheter have isolated a vein segment that contains a communicating vein.

FIG. 2 is a cross-sectional view of a vein having a catheter and introducer sheath inserted therein, wherein two substantially elastic expandable balloons carried on the end of the catheter have isolated a vein segment that does not contain a communicating vein.

FIG. 3 is a side view of a graduated scale printed along an outer surface of a catheter device according to one embodiment.

FIG. 4 is a cross-sectional view of a vein with a catheter inserted therein, wherein two substantially elastic balloons carried by the catheter are depicted in a non-inflated mode.

FIG. 5 is a cross-sectional view of the vein and inserted catheter of FIG. 4, with the balloons depicted in an inflated mode.

FIG. 6 is a profile view of a substantially elastic expandable balloon twisted along the axis of a catheter (shown in a non-inflated mode) according to one embodiment.

FIG. 7 is a profile view of the balloon of FIG. 6, when inflated.

FIG. 8 is a cross-sectional view of a vein with a catheter and introducer sheath inserted therein, wherein three, substantially elastic expandable balloons carried on a distal portion of the catheter have isolated two vein segments, one segment of which containing a communicating vein.

FIG. 9 is a cross-sectional view of a vein with a slidably adjustable catheter inserted therein, wherein two substantially elastic balloons carried by the catheter are depicted in an inflated mode.

### DETAILED DESCRIPTION OF THE DRAWINGS

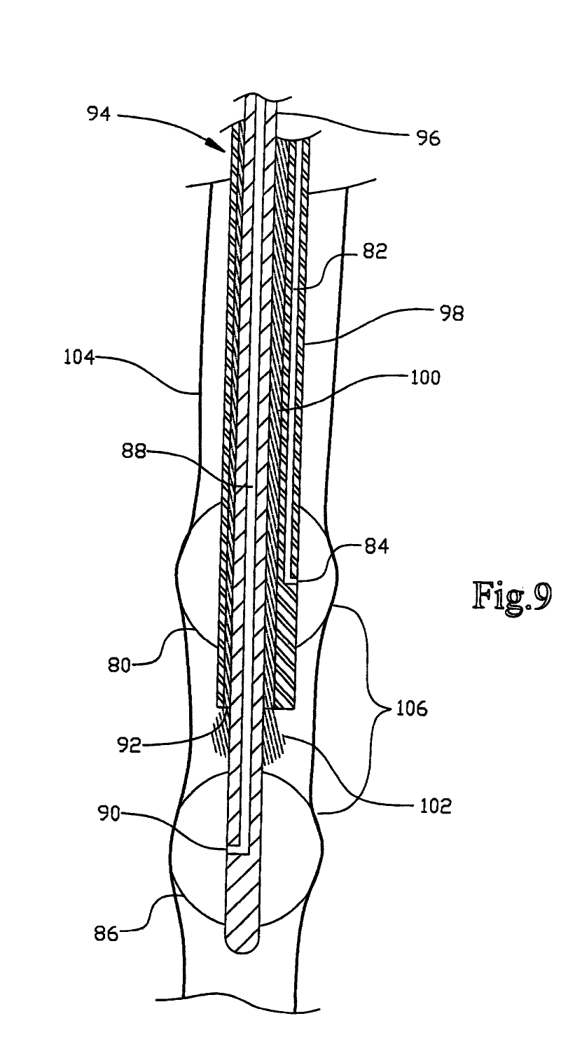

As used herein, “catheter” or “catheter device” refer to a generally tubular, flexible instrument for withdrawing or introducing fluids or performing diagnostic or therapeutic procedures within a duct, blood vessel, hollow organ or body cavity. However, the invention is not limited to a particular geometric cross-sectional shape (e.g., tubular), or construction (e.g., arrangement of lumens and/or steering mechanisms. Nor is the catheter or catheter device limited to a single body or member. For example, in the embodiment of FIG. 9 (discussed below), a “catheter” includes an inner catheter slidable within an outer catheter in a telescoping manner.

As used herein, “desired vein location” refers to a site in a patient's body where it is desirable for therapeutic reasons to locally deliver a medical agent. Once the general location has been selected, knowledge of the vascular system in that location will permit the user to select an appropriate venous access site and a catheter of the proper dimension to reach the desired vein location.

As used herein, “retrograde” refers to moving backward or against the usual direction of flow.

As used herein, “medical agent” refers to a “therapeutic agent” or a “diagnostic agent.”A “therapeutic agent” refers to any chemical or other material that is used in the treatment of a disease or disorder. Examples, without limitation, of therapeutic agents include gene therapy agents, antibiotics, antineoplastics, hormones, antivirals, radiation (via radiation sources such as cobalt, radium, radioactive sodium iodide, etc.), anticoagulants, enzymes, hepatoprotectants, vasodilators and the like. A therapeutic agent may also combined with another liquid such as physiologic saline or the like and may be administered using the devices and methods herein. A “diagnostic agent” refers to any chemical or other material that is used to determine the nature of a disease or disorder. Examples, without limitation, of diagnostic agents include dyes that react with metabolic products of a particular disease and radioactive materials that bind to and thereby indicate the presence of disease-causing entities within a patient's body.

As used herein, “imaging fluid” refers to any fluid composed of, or containing an agent that aids the use of various types of body scanners to distinguish tissue from surrounding tissues more easily. Examples, without limitation, include radiopaque contrast agents visible by x-ray systems, ferromagnetic or superparamagnetic metal particles visible by magnetic resonance imaging system, gas bubbles, low density or hollow spheres visible by ultrasonic imaging systems and the like.

As used herein, “marker” refers to markers used to visualize the location of the isolated vein segment or a distal tip of the catheter. Examples without limitation include radiopaque markers, visible by x-ray, and ferromagnetic or superparamagnetic markers, visible by magnetic resonance imaging.

As used herein, “serial vein” refers to any venous vessel that is part of a single blood flow path toward the heart.

As used herein, “communicating vein” refers to any venous vessel that provides more than one blood flow path toward the heart.

As used herein, “venule” refers to any small serial vein. The proximal end may connect with a vein or another venule and the distal end may connect to another venule or to capillaries.

As used herein, “substantially elastic” refers to a material that when used to make a balloon for a catheter of the present invention, provides at least a 25% recoverable expansion of the balloon diameter when inflated at the specified pressure.

As used herein, the term “about” means that the characteristic modified by such term may vary by as much as 20% from the norm for that characteristic and still be within the scope of this invention, unless expressly stated otherwise.

Preferred embodiments and implementations of the present invention are now described in conjunction with the accompanying figures.

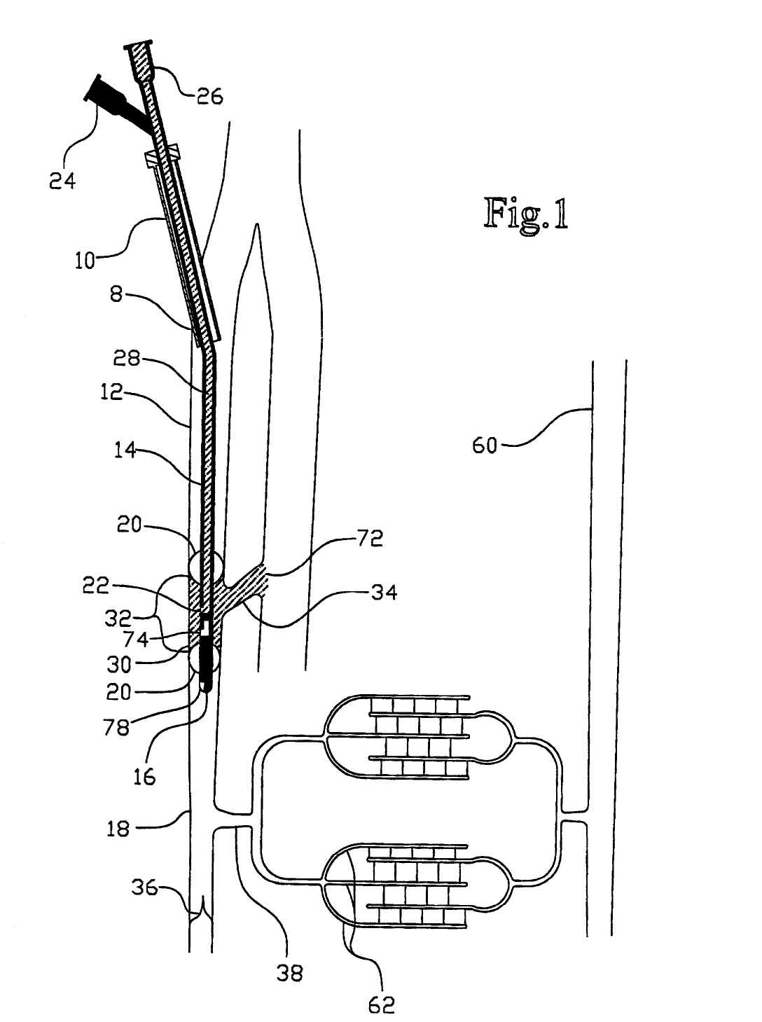

Referring to FIG. 1, an introducer sheath 10 has been inserted in a retrograde direction of a vein 12 at introduction site 8 providing access for a catheter 14. The catheter 14 has been inserted through the sheath 10 and into the vein 12, and subsequently advanced until a distal end 16 of the catheter 14 is located distal to a communication vein 34. The distal end 16 of the catheter 14 carries two substantially elastic expandable balloons 20 that are separated by sufficient distance to have a delivery port 22 in the body of the catheter between them. The balloons 20 are shown in an inflated state, forming a tight seal against a wall 30 of the vein 12, thus creating an isolated vein segment 32. Within the isolated vein segment 32, fluid is prevented from flowing between the surface of the expanded balloons 20 and the vein wall 30.

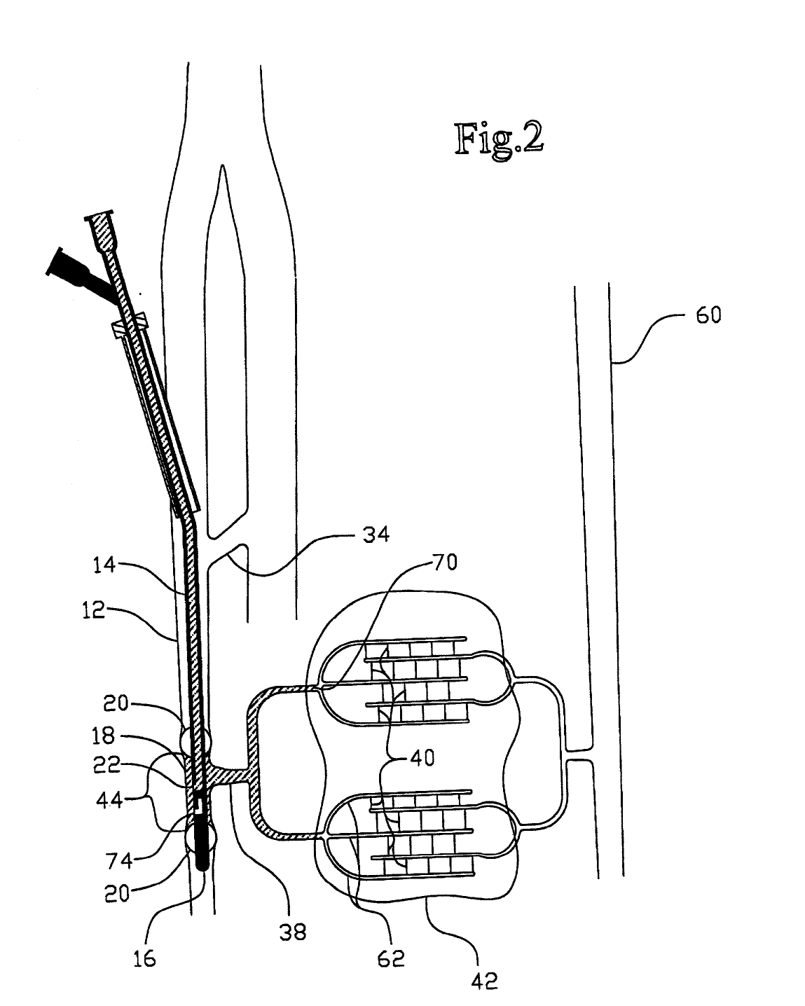

The balloons 20 are inflated with fluid injected through a luer fitting 24 that is connected to an inflation lumen not shown in the catheter 14, until the isolated vein segment 32 is able to withstand a pressure of at least 100 millimeters of mercury. In a preferred embodiment the balloons 20 are inflated until the isolated vein segment 32 is able to withstand a pressure of at least 150 and preferably 200 millimeters of mercury, without allowing any fluid leaking past the balloons 20. An imaging fluid 72 is advanced into the isolated vein segment 32 through the delivery port 22. The delivery port 22 and a luer fitting 26 are connected by a lumen 28. After the imaging fluid 72 is advanced into the isolated vein segment 32, a nearby communication vein 34 is viewed with a fluoroscope, magnetic resonance imaging system or other suitable imagining modality (not shown). If the imaging fluid 72 is seen flowing though the communication vein 34, then the vein segment isolation was not successful, and the balloons 20 are deflated. The catheter 14 is then advanced further into the vein 12, as seen in FIG. 2. Alternately, the catheter may be retracted in the vein 12 to avoid the common vein 34.

Once any valves have been detected, methods of advancing a catheter retrograde through veins containing valves may be employed to continue advancing the catheter through the venous system. Such methods are disclosed and described in U.S. patent application Ser. No. 09/595,853, entitled, “Methods of Catheter Positioning and Drug Delivery in Veins Containing Valves,” the disclosure of which is incorporated by reference.

Referring to FIG. 2, the catheter 14 has now been advanced until the distal end 16 is past the communication vein 34 and further into a desired vein location 18. The desired vein location 18 has a serial vein 38 that leads to a capillary system 40 within a desired treatment area 42. The balloons 20 are again inflated until an isolated vein segment 44 is able to withstand a pressure of at least 100 and preferably 200 millimeters of mercury without leaking past the balloons 20. Imaging fluid 70 is advanced into the isolated vein segment 44 through delivery port 22, where the imaging fluid 70 is viewed with a suitable imaging modality. If serial vein 38 is larger than about 0.05 millimeters, the imaging fluid 70 will be viewed as spreading past the serial vein 38 and into the desired treatment area 42. Veins smaller than 0.05 millimeters are usually not visible using current fluoroscopic imaging equipment.

After verifying that there are no communication veins within the isolated vein segment 44, a medical agent is advanced into the isolated vein segment 44, and into the desired treatment area 42. For example, the medical agent may be advanced into the isolated vein segment 44 through delivery port 22. Alternatively, the medical agent may be advanced into the isolated vein segment 44 through a separate delivery port (not shown) on the catheter 14.

Referring back to FIG. 1, there is a pressure measuring device 74 on the catheter 14 located between the balloons 20. The pressure measuring device is used to verify the pressure within the isolated vein segment 32.

In particular, the pressure measuring device 74 may be used in an alternative method for detecting the communicating vein 34 within the isolated vein segment 32. After the balloons 20 have been inflated, and the isolated vein segment 32 has been created, a pressure within the isolated vein segment is measured with the pressure measuring device 74. Fluid is then injected into isolated vein segment 32 though the delivery port 22, while a pressure differential caused within the isolated vein segment 32 is measured. Where a communicating vein 34 is present within the isolated vein segment 32 (shown in FIG. 1), the pressure differential will reflect a rise in pressure of less than about 100 millimeters of mercury. Where the isolated vein segment 44 does not have any communicating veins (shown in FIG. 2), there will be a higher pressure differential of about 100 to 1200 millimeters of mercury, since the injected fluid must overcome both an arterial back pressure created from an artery 60 and a dynamic pressure from the serial vein 38 and venules 62. Notably, the fluid viscosity and flow rate will have some influence the pressure differential.

Still referring to FIG. 1, in one embodiment there is a forward looking transducer 78 located on the distal tip 16 of the catheter 14. The forward looking transducer 78 is used to locate a valve 36 within the vein 12. Preferably, the transducer 78 is an ultrasound transducer.

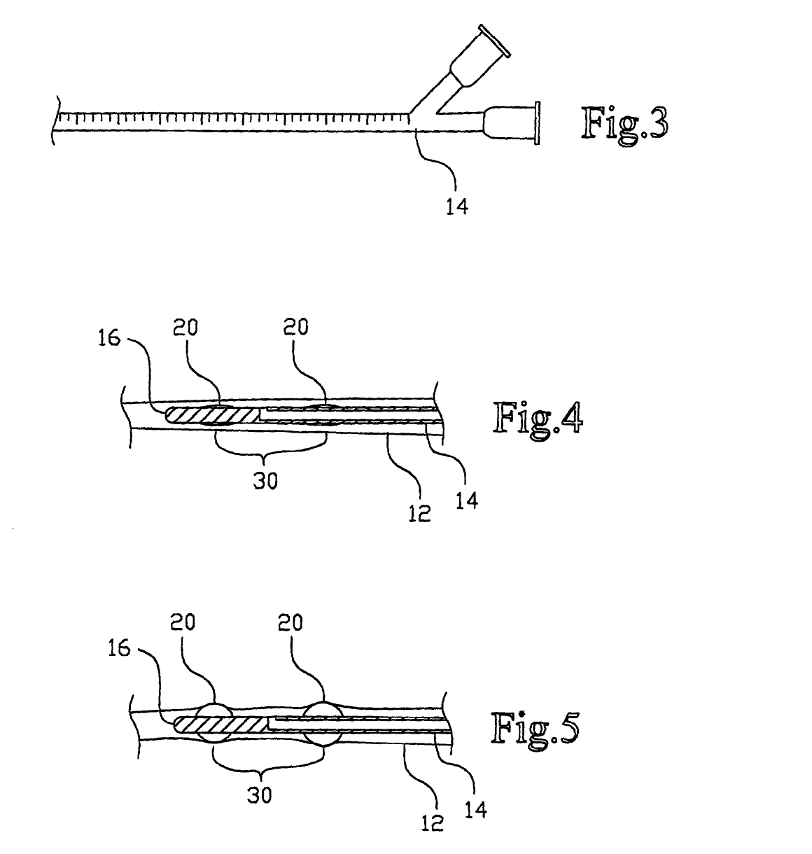

Referring to FIG. 3, in one embodiment, the catheter 14 has a graduated scale printed on the outside surface of the catheter 14. Referring to FIG. 1, a measurement is taken when the catheter 14 is initially put into the introducer sheath 10, measurements are subsequently recorded whenever a valve 36 or communicating vein 34 is encountered and when the desired vein location 18 without a communicating vein 34 is found. This is done in order to provide for repeatability and easy access whenever a replacement catheter has to be inserted into the same vein 12 at introduction site 8 for advancement into desired vein location 18.

For purposes of illustration, FIG. 4 shows the distal end 16 of the catheter 14 within the vein 12 with the balloons 20 in a deflated state. In FIG. 5, the balloons 20 are shown in an inflated state, where the elastic material has stretched to give a smooth balloon surface that forms a pressure tight seal to the vein wall 30 of the vein 12. Since the vein 12 is normally highly compliant, it is apparent when looking at the difference in FIGS. 4 and 5 that the vein will stretch in response to the pressure provided against the vein wall 30 by the inflated balloons 20.

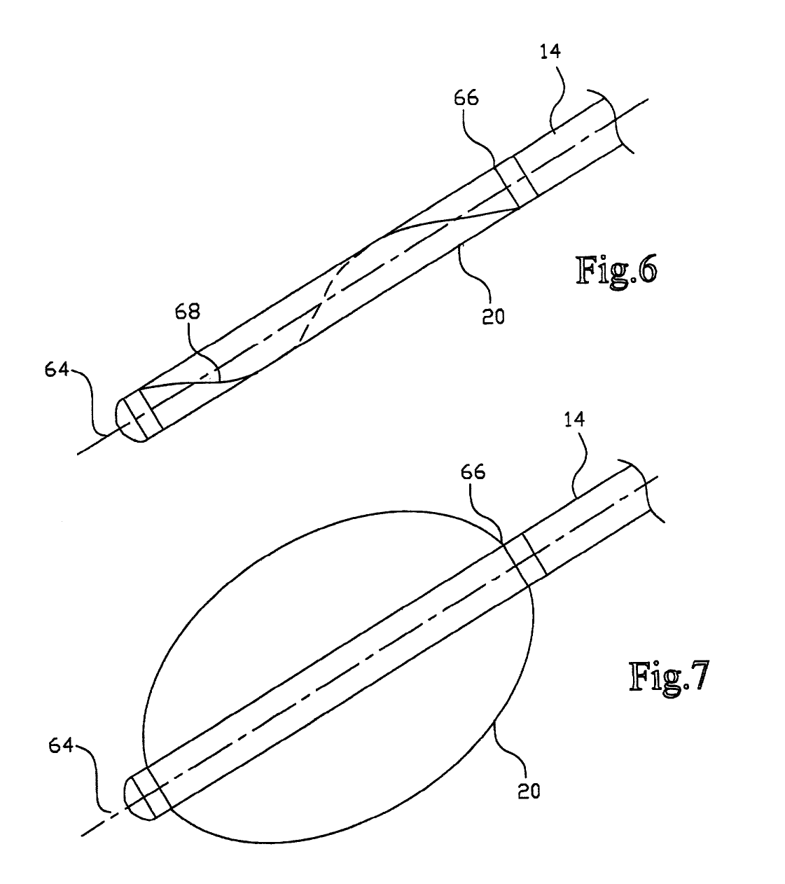

Referring to FIG. 6, in one embodiment, the balloon 20 is twisted along an axis 64 of the catheter 14 at a twist angle of about 20 degrees. Illustrated by line 68, a spiral twist was created when a proximal end 66 of the balloon 20 was attached to, and the balloon 20 was twisted along the axis 64 of the catheter 14. Wrinkles that would normally appear on the balloon 20 as a result of the spiral twist are not shown. Upon inflation, the balloon 20 will substantially take its normal shape and the spiral twist, represented by line 68, along with any wrinkles, will disappear, as shown in FIG. 7. During inflation, the spiral twist is stored within the balloon 20 as torque. Upon deflation of the balloon 20, the stored torque energy will return the balloon 20 substantially to its twisted form, as shown in FIG. 6.

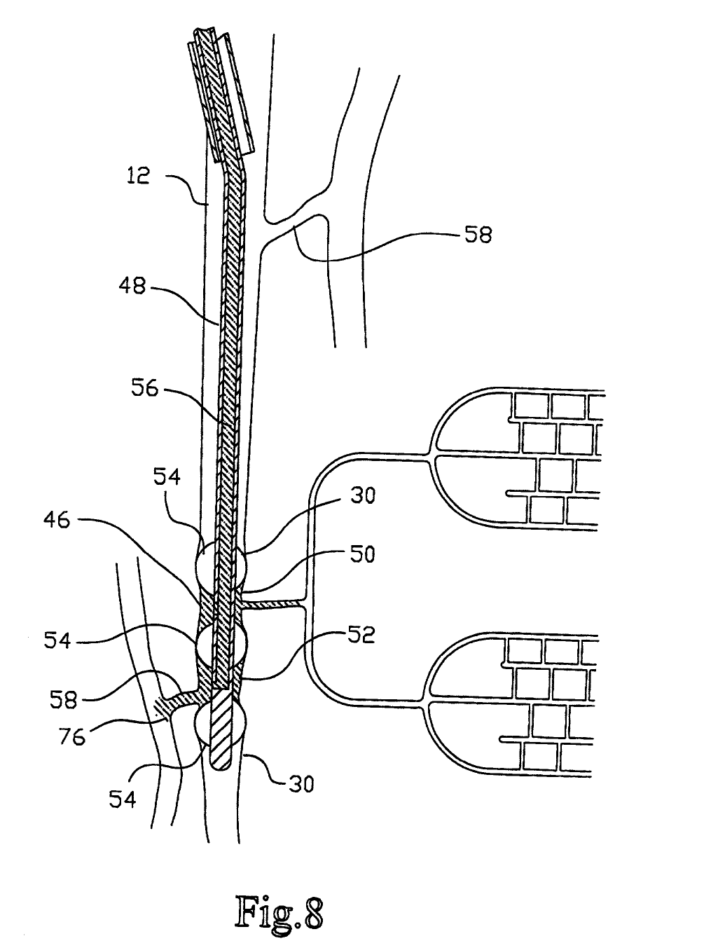

Referring to FIG. 8, in one embodiment, a catheter 48 is advanced to a desired vein location 46. The catheter 48 is configured to create two separate isolated vein segments, 50 and 52, at a desired vein location 46. The isolated vein segments 50 and 52 are created by three vein occlusion devices 54 expanded to engage the vein wall 30 of the vein 12, thereby blocking the interior of the vein 12 and preventing fluid from flowing between the respective vein occlusion devices 54 and the vein wall 30. The catheter 48 also incorporates a lumen 56 to direct an imaging fluid 76 or medical agent through the catheter 48 and into the isolated vein segments 50 and 52. By forming multiple vein segments 50 and 52, this embodiment increases the probability that at least one segment will not contain any communicating veins. This design allows the placement of the catheter 48 more quickly since it eliminates the need to inflate the vein occlusion devices 54, check for leakage through a communicating vein 58, relocate the catheter 48 if necessary and recheck for the communicating vein 58.

In a preferred embodiment, each vein occlusion device 54 comprises a substantially elastic expandable balloon. If the substantially elastic expandable balloon material is perfectly compliant, an inflation pressure of at least 100 millimeters of mercury would be required to maintain a pressure of 100 millimeters of mercury within the isolated vein segment. Since a perfectly compliant balloon is not possible, the balloon inflation pressure actually has to be somewhat higher than the required pressure within the isolated vein segment.

Unlike arteries, the veins are highly compliant. This is due to lower amounts of elastic tissue, smooth muscle cells, and fibrous tissue in veins compared to arteries. This also gives the veins an ability to undergo large volume changes when subjected to small changes in pressure. For example, veins may double in volume when exposed to pressure increases of 30 to 60 millimeters of mercury. As a result, the design of an expandable balloon for use in veins is different from an expandable balloon for use in an artery. For use in a vein, the balloon must be substantially elastic to accommodate the high elasticity and expansion seen in veins. Since some communicating veins are very small, an injection pressure of 100 millimeters of mercury or greater should be used when injecting contrast solution to properly visualize all of the communicating veins. In order to insure a good seal between the balloons and the elastic vein wall, a balloon inflation pressure of at least 200 millimeters of mercury should be used. This significantly limits the choices available for the balloon material and design. The balloon must be thin in order to collapse small enough to access small veins yet have a combination of high elasticity and high strength.

It has been found that a substantially elastic material will stretch to give a smooth balloon surface that will form a pressure tight seal to the wall of the expanding vein upon inflation. Since the substantially elastic material will stretch while expanding, the balloon is able to form a pressure tight seal within a wider range of vein sizes as compared to a less elastic material that needs to be almost fully inflated in order to form a pressure tight seal. Also, the substantially elastic material will return to its original shape upon deflation, while a less elastic material is more subject to deformation and creasing.

In accordance with another embodiment shown in FIG. 9, an adjustable (i.e., telescoping) catheter distal tip assembly 94 includes an inner catheter 96 slidebly disposed within a lumen 100 of an outer catheter 98. Within inner catheter 96 is an inflation lumen 88 that extends from a luer fitting (not shown) at the proximal end of inner catheter 96, terminating at a port 90 within an inflatable, elastic balloon 86. The balloon 86 is inflatable with fluid (as shown in FIG. 9), which is injected through the inflation lumen 88. In a preferred embodiment, the inflated balloon 86 is able to withstand a pressure of at least 100 millimeters of mercury. While not shown in FIG. 9, it will be apparent that inner catheter 96 can include additional lumens besides lumen 88, useful for providing access for steerable guide wires, forward looking transducers, etc.

Within outer catheter 98 is an inflation lumen 82 that extends from a luer fitting (not shown) at the proximal end of outer catheter 98 and terminates at port 84 within an inflatable, elastic balloon 80. The balloon 80 is inflatable with fluid (as shown in FIG. 9), which is injected through inflation lumen 82 and is preferably able to withstand a pressure of at least 100 millimeters of mercury. It will be appreciated that balloons 80 and 86 can also incorporate the spiral twist structure of balloon 20 of FIG. 6.

In accordance with this embodiment, the inner catheter 96 may be slidably advanced or retracted within the outer catheter 96, allowing the user to adjust the spacing between balloons 80 and 86, while the catheter assembly 94 is placed within a patient's blood vessel 104. The ability to independently position either balloon provides more flexibility in forming an isolated vein segment 106 in a vein location that does not contain any communicating vein branches. After inflating balloons 80 and 86 to form isolated vein segment 106, and then verifying that isolated vein segment 106 does not contain any communicating vein branches, a imaging or medical agent 102 may be directed through lumen 100 of outer catheter 98, exiting at its distal end 92 and into the isolated vein segment 106.

While the invention has been described and explained in the context of the preferred embodiments discussed above, it will be understood by those skilled in the art that various changes may be made to those embodiments, and various equivalents may be substituted, without departing from the scope of the invention as defined only by the appended claims and their equivalents.

What is claimed:

1. Apparatus for creating, and delivering a medical agent into, an isolated vein segment, the apparatus comprising:
a catheter carrying a pair of spaced apart expandable occlusion devices along a distal portion, the catheter configured for insertion and advancement in a venous system such that the occlusion devices may be positioned at a desired location in a vein,
the occlusion devices expandable to engage and expand a wall of the vein, thereby isolating an interior segment of the vein between the occlusion devices and preventing fluid located in the isolated vein segment past the respective occlusion devices and vein wall at a pressure within the isolated vein segment of at least 100 millimeters of mercury,
the catheter comprising one or more lumens for directing a fluid into the isolated vein segment at a pressure of at least 100 millimeters of mercury.

2. The apparatus of claim 1, the occlusion devices comprising inflatable balloons.

3. The apparatus of claim 2, the inflatable balloons comprising a material with a hardness of between Shore 25D and 55D, and a flexural modulus of elasticity of between 500 and 2500 pounds per square inch.

4. The apparatus of claim 2, the catheter comprising a shaft with an axis, the inflatable balloons twisted along the axis of the catheter shaft when not inflated so as to maintain a relatively low profile, allowing for navigation of the catheter through the venous system.

5. The apparatus of claim 4, wherein the balloons are twisted along the axis of the catheter shaft at a twist angle of about 20 degrees.

6. The apparatus of claim 1, the catheter carrying a forward looking transducer positioned to detect valves or other obstructions as the catheter is navigated through the venous system.

7. The apparatus of claim 6, wherein the transducer is an ultrasound transducer.

8. The apparatus of claim 1, further comprising a pressure measurement device configured to measure a pressure within the isolated vein segment.

9. The apparatus of claim 8, wherein the pressure measurement device is carried on the catheter between the occlusion devices.

10. The apparatus of claim 1, further comprising a measuring system for measuring a distance that the catheter has traveled within the venous system.

11. The apparatus of claim 10, the measuring system comprising a graduated scale located on an outer surface of the catheter.

12. The apparatus of claim 1, the catheter carrying a plurality of markers, including a first marker on a distal end of the catheter, and a marker on each occlusion device.

13. The apparatus of claim 1, the catheter carrying at least three spaced apart occlusion devices, whereby at least two separate vein segments may be created by expanding the respective occlusion devices.

14. The apparatus of claim 1, wherein the catheter comprises an inner catheter member slidably disposed in an interior lumen of an outer catheter member, a distal portion of the inner catheter member extending beyond a distal end of the outer catheter member, a first occlusive device disposed on the distal portion of the inner catheter member, a second occlusive device disposed on a distal portion of the outer catheter member, whereby a length of an isolated vein segment between the first and second occlusive devices may be determined by sliding the inner catheter member relative to the outer catheter member.

15. The apparatus of claim 14, the first and second occlusive devices comprising respective first and second inflatable balloons, the inner catheter member having an inflation lumen in communication with the first inflatable balloon, the outer catheter member having an inflation lumen in communication with the second inflatable balloon.

16. A method for delivering a medical agent in a venous system, comprising:
advancing a catheter through the venous system;
isolating an interior segment of a vein;
verifying that the isolated vein segment does not contain any communicating vein branches; and
directing a medical agent through the catheter and into the isolated vein segment.

17. The method of claim 16, the isolating step comprising
expanding a pair of spaced apart occlusion devices carried by the catheter to engage and expand a wall of the vein, thereby preventing fluid located within the isolated segment from flowing past the respective occlusion devices and vein wall at a pressure within the isolated vein segment of at least 100 millimeters of mercury.

18. The method of claim 17, the verifying step comprising injecting a fluid into the isolated vein segment at a pressure of at least 100 millimeters of mercury.

19. The method of claim 18, the fluid comprising an imaging fluid, the verifying step further comprising imaging the isolated vein segment while injecting the fluid therein.

20. The method of claim 18, the verifying step comprising measuring a pressure within the isolated vein segment while injecting the fluid therein.

21. The method of claim 16, the isolating step comprising creating at least two isolated vein segments; the directing step comprising directing a medical agent through the catheter into at least one of the at least two isolated vein segments.

22. The method of claim 21, wherein the at least two isolated vein segments are created by
expanding at least three spaced apart occlusion devices carried by the catheter to engage and expand a wall of the vein, thereby preventing fluid located within any isolated vein segment from flowing past the respective occlusion devices and vein wall at a pressure within the respective isolated segment of at least 100 millimeters of mercury.
